(19) United States
(12) Patent Application Publication
(10) Pub. No.: US 2007/0254001 A1
(43) Pub. Date: Nov. 1, 2007

(54) APPARATUS FOR MANUFACTURING THREE-DIMENSIONAL MATERIAL FOR REGENERATION OF TISSUE AND MANUFACTURING METHOD USING THE SAME

(75) Inventors: Geun-hyung KIM, Daejeon (KR); Wan-doo KIM, Daejeon (KR)

Correspondence Address: CANTOR COLBURN, LLP, 55 GRIFFIN ROAD SOUTH, BLOOMFIELD, CT, 06002, US

(73) Assignee: KOREA INSTITUTE OF MACHINERY & MATERIALS, Daejeon (KR)

(21) Appl. No.: 11/617,851

(22) Filed: Dec. 29, 2006

(30) Foreign Application Priority Data
Apr. 26, 2006 (KR) 1020060037521

(51) Int. Cl. A61L 33/00; A61F 13/00

(52) U.S. Cl. 424/422; 427/2.24; 118/668

(57) ABSTRACT

Disclosed is an apparatus for manufacturing a three-dimensional material for the regeneration of a tissue in which basic frameworks are formed by use of a plotter and nano-fibrous cell culture scaffolds are formed in the interior of and on the exterior surface of the basic framework, so as to achieve a reduction in the manufacturing time of the three-dimensional material for the regeneration of a tissue, and a manufacturing method using the same. The apparatus for manufacturing the three-dimensional material, which will be inserted into the human body and used for the regeneration of a tissue, includes the plotter for forming the three-dimensional basic frameworks, an electric radiator for forming the nano-fibrous cell culture scaffolds between the three-dimensional basic frameworks as well as on the surface of the respective basic frameworks, and a control computer for controlling operations of the plotter and the electric radiator.

### BACKGROUND OF THE INVENTION

1. Field of the Invention

The present invention relates to an apparatus for manufacturing a three-dimensional material for the regeneration of a tissue and a manufacturing method using the same, and more particularly, to an apparatus for manufacturing a three-dimensional material, more particularly, cell culture scaffolds, for the regeneration of a tissue, in which three-dimensional basic frameworks are formed by use of a plotter and the cell culture scaffolds, in the form of nano-scale fibers, are formed between the basic frameworks as well as on the surface of the respective basic frameworks, whereby the manufacturing time of the three-dimensional nano-fibrous scaffolds for the regeneration of a tissue can be reduced, and a manufacturing method using the above described apparatus.

2. Description of the Related Art

If internal organs or tissues of the human body are damaged, cells, medicinal scaffolds, etc. are used to efficiently regenerate the damaged tissues. Scaffolds for the regeneration of tissues must have a physiological activity for showing a physical stability in implant regions and regulating the efficacy of regeneration. Also, after completing the regeneration of new tissues, the scaffolds have to be resolved in the body. In this case, a resulting resolved product must have no toxicity.

Conventional scaffolds for the regeneration of tissues may be classified into a sponge type cell culture scaffold using a polymer having a predetermined strength and shape, a nano-fibrous matrix type cell culture scaffold, or a gel type cell culture scaffold. These cell culture scaffolds have an important role of making a three-dimensional tissue having a specific depth or height.

A technology for implanting a scaffold functioning as a framework for use in the regeneration of a tissue and regenerating the tissue in the living body by using a self-healing power of the living body is called “Regenerative Medicine” or “Tissue Engineering”.

One example of the Tissue Engineering is a method for regenerating a joint cartilage. In the joint cartilage regenerating method, after forming an artificial prosthesis that uses a cartilage cell as a scaffold, the artificial prosthesis is implanted into a damaged joint region of the body so that the cartilage cell is regenerated in the damaged joint region.

The artificial prosthesis includes three-dimensional scaffolds, which are formed by use of cartilage cells, etc. as a seed.

As a method for forming the three-dimensional scaffolds, conventionally, a rapid prototyping method, more particularly, lamination rapid prototyping method, has been used.

In the lamination rapid prototyping method, to obtain a desired shape of a completed article, after processing a plurality of individual sheets in the form of a multilayer structure, the sheets are laminated sequentially to have the desired shape. More particularly, a three-dimensional object, which is modeled by use of a CAD system, is divided into a plurality of sheets having a predetermined thickness and converted into slice data. Thereafter, on the basis of the slice data, the sheets in the form of plates are prototyped and laminated sequentially, to manufacture a prototyped object.

In the above described prior art method for manufacturing a three-dimensional material for the regeneration of a tissue in which a three-dimensional object is modeled in a predetermined manner and is divided into a plurality of sheets having a predetermined thickness and then, the plurality of divided sheets are laminated sequentially, however, there is a problem in that an excessively long time is required for the preparation and lamination of divided sheets.

### SUMMARY OF THE INVENTION

Therefore, the present invention has been made in view of the above problems, and it is an object of the present invention to provide an apparatus for manufacturing a three-dimensional material for the regeneration of a tissue, in which three-dimensional basic frameworks are formed by use of a bio-plotter and then, nano-fibrous scaffolds are formed between the basic frameworks as well as on the surface of the respective basic frameworks by use of an electric radiator, whereby the three-dimensional material for the regeneration of a tissue can be manufactured within a short time and the scaffolds for the propagation of cells can achieve a nano-scale structure, and a manufacturing method using the above described apparatus.

In accordance with an aspect of the present invention, the above and other objects can be accomplished by the provision of an apparatus for manufacturing a three-dimensional material that will be inserted into the human body and used for the regeneration of a tissue, the apparatus comprising: a plotter for forming three-dimensional basic frameworks; an electric radiator for forming cell culture scaffolds, in the form of nano-scale fibers, between the three-dimensional basic frameworks as well as on the surface of the respective basic frameworks formed by the plotter; and a control computer for controlling operations of the plotter and the electric radiator.

Preferably, the plotter and the electric radiator are integrally formed with each other.

Preferably, the electric radiator comprises: a solution storage tank for storing a bio-polymer solution therein; a nozzle installed to inject the solution supplied from the storage tank by electric radiation; a collector provided below the nozzle and adapted to allow the solution radiated from the nozzle to be accumulated on a surface thereof to have the form of fibers; and a voltage generator connected between the nozzle and the collector and adapted to apply a voltage to both the nozzle and the collector. Preferably, the plotter is configured to be movable in X, Y, and Z-axes directions under the operation of a motor and comprises a plotter nozzle adapted to discharge a solution supplied from a solution storage tank storing a bio-polymer solution therein.

In accordance with another aspect of the present invention, the above and other objects can be accomplished by the provision of a method for manufacturing a three-dimensional material for the regeneration of a tissue comprising: forming basic frameworks having a three-dimensional shape by use of a plotter that is movable in X, Y, and Z-axes directions; and forming nano-fibrous cell culture scaffolds, via an electric radiation manner, between the three-dimensional basic frameworks as well as on the surface of the respective basic frameworks formed by the plotter.

Preferably, the formation of the three-dimensional basic frameworks comprises: generating three-dimensional data via modeling of the three-dimensional shape; generating plotter data from the three-dimensional data; and moving the plotter in X, Y, and Z-axes directions on the basis of the plotter data, to laminate the basic frameworks in multiple layers

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a configuration block diagram of an apparatus for manufacturing a three-dimensional material for the regeneration of a tissue according to the present invention;

FIG. 2 is an exploded perspective view illustrating an embodiment of the apparatus for manufacturing a three-dimensional material for the regeneration of a tissue according to the present invention; and

FIG. 3 is an exploded perspective view illustrating an embodiment of a plotter shown in FIG. 2.

### DETAILED DESCRIPTION OF THE PREFERRED EMBODIMENTS

Now, preferred embodiments of the present invention will be described with reference to the accompanying drawings.

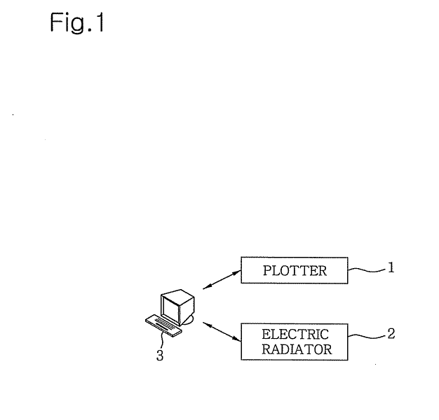

FIG. 1 is a configuration block diagram of an apparatus for manufacturing a three-dimensional material for the regeneration of a tissue according to the present invention. The apparatus of the present invention is used to manufacture a three-dimensional material, which will be inserted into the human body for the regeneration of a tissue. The apparatus for manufacturing the three-dimensional material for the regeneration of a tissue comprises a plotter 1 for forming three-dimensional basic frameworks, an electric radiator 2 for forming nano-fibrous cell culture scaffolds between the three-dimensional basic frameworks as well as on the surface of the respective basic frameworks formed by the plotter 1, and a control computer 3 for controlling the overall operations of the plotter 1 and the electric radiator 2.

The plotter 1 and the electric radiator 2 are integrally formed with each other. The plotter 1 is movable in X, Y, and Z-axes directions.

Specifically, the plotter 1 is backed up by the computer 3, which has the function of designating X, Y, and Z-axes directional positions of the plotter 1, so as to designate the address of the plotter 1 for the formation of the three-dimensional basic framework.

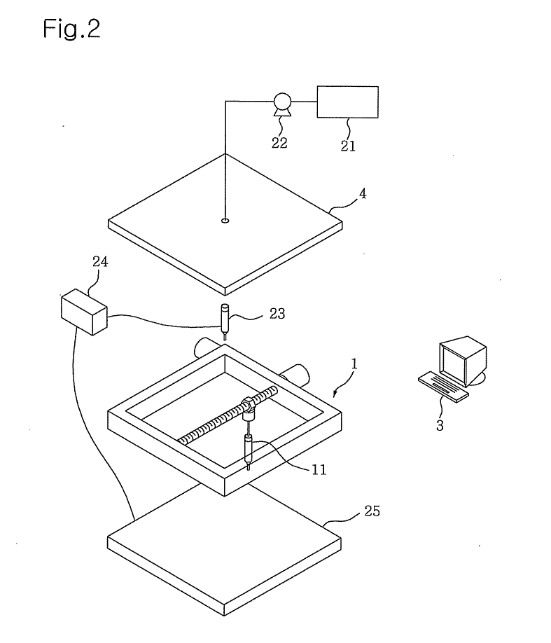

FIG. 2 is an exploded perspective view illustrating an embodiment of the apparatus for manufacturing the three-dimensional material for the regeneration of a tissue according to the present invention. Referring to FIG. 2, the apparatus for manufacturing the three-dimensional material for the regeneration of a tissue according to the present invention further comprises a stage 4, to which a nozzle 23 of the electric radiator 2 is installed. The electric radiator 2 is adapted to inject the nano-fibrous cell culture scaffolds through the nozzle 23 via an electric radiation manner. The plotter 1 has a plotter nozzle 11, which is coupled to the stage 4 and adapted to receive a solution supplied from a solution storage tank of the electric radiator 2 that will be described hereinafter. With this configuration, the plotter 1 is adapted to inject the solution through the plotter nozzle 11, so as to form the framework of the three-dimensional material for the regeneration of a tissue.

The electric radiator 2 includes a solution storage tank 21 for storing a bio-polymer solution to be supplied into the nozzle 23, a collector 25 provided below the nozzle 23 for allowing the solution radiated from the nozzle 23 to be accumulated on a surface thereof to have the form of nano-scale fibers, and a voltage generator 24 connected between the nozzle 23 and the collector 25 for applying a voltage to both the nozzle 23 and the collector 25.

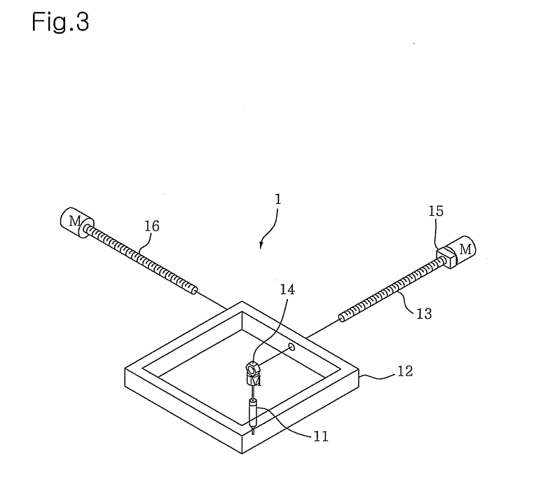

FIG. 3 is an exploded perspective view illustrating an embodiment of the plotter 1 shown in FIG. 2. The plotter 1 is installed so that it can move in X, Y, and Z-axes directions under the operation of a motor (not shown). As shown in the drawing, the plotter 1 includes a frame 12 provided below the stage 4 (See FIG. 2), a screw shaft 13 penetrated through opposite side portions of the frame 12 and adapted to move forward and rearward of the frame 12 under the operation of the motor, a nut 14 fastened on the screw shaft 13 and adapted to move in a longitudinal direction of the screw shaft 13 under the operation of the motor, and an auxiliary screw shaft 16 bearing coupled to the screw shaft 13 within a block 15 and adapted to move not only the plotter nozzle 11, which is located below the nut 14 and adapted to be moved up and down under the operation of the motor, but also the screw shaft 13, forward and rearward of the frame 12 under the operation of the motor.

Preferably, the control computer 3 includes a three-dimensional data preparing unit for preparing three-dimensional data related to the material for the regeneration of a tissue, a plotter data generating unit for generating plotter data from the three-dimensional data, and an output unit for outputting signals for controlling the operations of the plotter 1 and the electric radiator 2.

Hereinafter, a manufacturing method using the above described apparatus for manufacturing the three-dimensional material for the regeneration of a tissue will be described with reference to FIGS. 1 to 3.

First, a three-dimensional shape of an artificial prosthesis is molded by use of the three-dimensional data preparing unit provided in the control computer 3, to prepare three-dimensional data. Then, on the basis of the three-dimensional data, plotter data is generated.

Subsequently, the control computer 3 outputs a drive signal to the plotter 1 on the basis of the plotter data. As the plotter 1 moves in X, Y, and Z-axes directions in response to the drive signal, a solution is injected through the plotter nozzle 11, to form a basic framework.

After completing the formation of the basic frameworks, the electric radiator 2 is operated, to form nano-fibrous cell culture scaffolds between the basic frameworks as well as the surface of the respective basic frameworks formed by the plotter 1.

Specifically, if a high voltage is applied between the nozzle 23 and the collector 25 and the solution stored in the solution storage tank 21 is supplied into the nozzle 23, the solution is able to be radiated from the nozzle 23, thereby being accumulated in the basic framework formed by the plotter 1 to have the form of nano-scale fibers.

Consequently, according to the present invention related to the manufacture of the three-dimensional material for the regeneration of a tissue, the three-dimensional basic frameworks are formed by use of the plotter having the plotter nozzle, and the nano-fibrous cell culture scaffolds are formed between the basic frameworks as well as on the surface of the respective basic frameworks formed by the plotter by use of the electric radiator.

As apparent from the above description, the present invention provides an apparatus for manufacturing a three-dimensional material for the regeneration of a tissue, which comprises a plotter having a plotter nozzle adapted to discharge a bio-polymer solution, the plotter being movable in X, Y, and Z-axes directions and used to form three-dimensional basic frameworks, and an electric radiator for forming nano-fibrous cell culture scaffolds between the basic frameworks as well as on the surface of the respective basic frameworks. The manufacturing apparatus having the above described configuration has the effect of reducing the manufacturing time of the three-dimensional nano-fibrous scaffolds for the regeneration of a tissue, and of achieving an improvement in the propagation efficiency of cells by virtue of the nano-scale cell culture scaffolds.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. An apparatus for manufacturing a three-dimensional material that will be inserted into the human body and used for the regeneration of a tissue, the apparatus comprising:
a plotter for forming three-dimensional basic frameworks;
an electric radiator for forming cell culture scaffolds, in the form of nano-scale fibers, between the three-dimensional basic frameworks as well as on the surface of the respective basic frameworks formed by the plotter; and
a control computer for controlling operations of the plotter and the electric radiator.

2. The apparatus according to claim 1, wherein the plotter and the electric radiator are integrally formed with each other.

3. The apparatus according to claim 2, wherein the electric radiator comprises:
a solution storage tank for storing a bio-polymer solution therein;
a nozzle installed to inject the solution supplied from the storage tank by electric radiation;
a collector provided below the nozzle and adapted to allow the solution radiated from the nozzle to be accumulated on a surface thereof to have the form of fibers; and
a voltage generator connected between the nozzle and the collector and adapted to apply a voltage to both the nozzle and the collector.

4. The apparatus according to claim 2, wherein the plotter is configured to be movable in X, Y, and Z-axes directions under the operation of a motor and comprises a plotter nozzle adapted to discharge a solution supplied from a solution storage tank storing a bio-polymer solution therein.

5. The apparatus according to claim 1, wherein the electric radiator comprises:
a solution storage tank for storing a bio-polymer solution therein;
a nozzle installed to inject the solution supplied from the storage tank by electric radiation;
a collector provided below the nozzle and adapted to allow the solution radiated from the nozzle to be accumulated on a surface thereof to have the form of fibers; and
a voltage generator connected between the nozzle and the collector and adapted to apply a voltage to both the nozzle and the collector.

6. The apparatus according to claim 1, wherein the plotter is configured to be movable in X, Y, and Z-axes directions under the operation of a motor and comprises a plotter nozzle adapted to discharge a solution supplied from a solution storage tank storing a bio-polymer solution therein.

7. A method for manufacturing a three-dimensional material for the regeneration of a tissue comprising:
forming basic frameworks having a three-dimensional shape by use of a plotter that is movable in X, Y, and Z-axes directions; and
forming nano-fibrous cell culture scaffolds, via an electric radiation manner, between the three-dimensional basic frameworks as well as on the surface of the respective basic frameworks formed by the plotter.

8. The method according to claim 7, wherein the formation of the three-dimensional basic frameworks comprises:
generating three-dimensional data via modeling of the three-dimensional shape;
generating plotter data from the three-dimensional data; and
moving the plotter in X, Y, and Z-axes directions on the basis of the plotter data, to laminate the basic frameworks in multiple layers.
